(19) United States
(12) Patent Application Publication
(10) Pub. No.: US 2003/0031653 A1
(43) Pub. Date: Feb. 13, 2003

(54) Compositions and methods for eliciting immune responses with a secretion-directed protein

(76) Inventors: Patrick J. Farrell, Calgary (CA); Lashitew Gedamu, Clagary (CA); Kostas Iatrou, Clagary (CA)

Correspondence Address: BURNS DOANE SWECKER & MATHIS L L P, POST OFFICE BOX 1404, ALEXANDRIA, VA, 22313-1404, US

(21) Appl. No.: 09/925,287

(22) Filed: Aug. 8, 2001

(51) Int. Cl. A61K 48/00; A61K 38/19; A61K 38/20

(52) U.S. Cl. 424/93.21; 424/85.1; 514/44

(57) ABSTRACT

The present invention relates to the use of nucleic acids which code for secretory fusion proteins to elicit immune responses. The secretory fusion protein comprises a signal sequence from a secretion competent protein, particularly a cytokine, which is linked in frame with the coding sequence of the antigen of interest which is normally intracellular. The nucleic acid encoding such a fusion protein is significantly more effective in its ability to elicit antibody production than the nucleic acid encoding the intracellular antigen. The present invention thus provides for a method of generating antibodies as well as a method of eliciting protective immune response using such nucleic acids. Also provided are pharmaceutical compositions comprising these nucleic acids.

### FIELD OF THE INVENTION

The present invention relates to the use of a protein secretion module to enhance secretion of a protein and, in particular, compositions and methods for enhancing humoral immune responses to the protein.

### REFERENCES

U.S. Pat. No. 6,037,150.

U.S. Pat. No. 6,194,388.

WO00/50616.

Alberts et al. 1994 (3^{rd} ed.). Molecular Biology of the Cell. Garland Publishing, Inc. (New York & London).

Bany, B. M., and G. A. Schultz. 2001. Increased expression of a novel heat shock protein transcript in the mouse uterus during decidulaization and in response to progesterone. Biology of Reproduction 64: 284-292.

Farrell, P. J., Behie, L. A., and K. Iatrou. 2000. Secretion of Intracellular Proteins from Animal Cells using a Novel Secretion Module. Proteins—Structure, Function, and Genetics 41:144-150.

Farrell, P. J., Lu, M., Prevost, J., Brown, C., Behie, L. A., and K. Iatrou. 1998. High Level of Expression Recombinant Glycoproteins from Transformed Lepidopteran Insect Cells Using a Novel Expression Vector. Biotechnology and Bioengineering 60: 656-663.

Gough, N. M., Metcalf, D., Gough, J., Grail, D. and Dunn, A. R. 1985. Structure and expression of the mRNA for murine granulocyte-macrophage colony stimulating factor. EMBO J. 4: 645-653.

Hammock, B. D., Bonning, B. B., Possee, R. D., Hanzlik, T. N., and S. Maeda. 1990. Expression and the effects of juvenile hormone esterase in a baculovirus vector. Nature 344: 458-461.

Hromas, R., Kim, C. H., Klemsz, M., Krathwohl, M., Fife, K., Cooper, S., Schnizlein-Bick, C. and Broxmeyer, H. E. 1997. Isolation and characterization of Exodus-2, a novel C-C chemokine with a unique 37-amino acid carboxyl-terminal extension. J. Immunol. 159: 2554-2558.

Johnson, R., Meidinger, R. G., and K. Iatrou. 1992. A cellular promoter-based expression cassette for generating recombinant baculoviruses directing rapid gene expression in passenger genes in infected insects. Virology 190: 815-823.

Kashima, N., Nishi-Takaoka, C., Fujita, T., Taki, S., Yamada G., Hamuro, J. and Taniguchi, T. 1985 Unique structure of murine interleukin-2 as deduced from cloned cDNAs. Nature 313: 402-404.

Liljeqvist, S. and S. Stahl. 1999. Production of recombinant subunit vaccines: protein immunogens, live delivery systems and nucleic acid vaccines. J. Biotechnol. 73: 1-33.

Ma, D., Lawless, D. and Riabowol, K. 1999. Sequence conservation of ING1 splicing isoforms in divergent species. Nat. Genet. 23: 373.

Ngo, V. N., Tang, H. L. and Cyster, J. G. 1998. Epstein-Barr virus-induced molecule 1 ligand chemokine is expressed by dendritic cells in lymphoid tissues and strongly attracts naive T cells and activated B cells. J. Exp. Med. 188: 181-191.

Opyene, A. L. O. and L. Gedamu. 1997. Molecular cloning, characterization and overexpression of two distinct cysteine protease cDNA's from Leishmania chagasi. Mol. Biochem. Parasitol. 90: 247-267.

Schoenhaut, D. S., Chua, A. O., Wolitzky, A. G., Quinn, P. M., Dwyer, C. M., Gately, M. K. and Gubler, U. 1992. Cloning and Expression of Murine IL-12. J. Immunol. 148: 3433-3440.

Sideras, P., Bergstedt-Lindquist, S., Severinson, E., Noma, Y., Naito, T., Azuma, C., Tanabe, T., Kinashi, T., Matsude, F., Yaoita, Y., and Honjo, T. 1987. IgG1 induction factor: A single molecular entity with multiple biological functions. Adv. Exp. Med. Biol. 213: 227-236.

Svanholm, C. et al. 1999. enhancement of antibody responses by DNA immunization using expression vectors mediating efficient antigen secretion. J. Immunological Methods 228: 121-130.

Ulmer et al. 1993. Heterologous protection against influenza by injection of DNA encoding a viral protein. Science 259: 1745-1749.

All of the above publications, patents and patent applications are herein incorporated by reference in their entirety to the same extent as if the disclosure of each individual publication, patent application or patent was specifically and individually indicated to be incorporated by reference in its entirety.

### BACKGROUND OF THE INVENTION

Using naked DNA for vaccination was first reported in 1993 when Ulmer et al. described a technique to induce a protective immune response in mice using a gene from influenza A virus. In this technique, DNA expression plasmids containing the gene for the protein antigen are usually injected intramuscularly in saline solution where they are taken up into muscle cells. Once inside the cells, the encoded antigen is expressed under control of a mammalian promoter, and cytoxic T-lymphocyte (CTL), T helper (Th) responses, and humoral (antibody) immunity to the encoded antigen can result.

Over 50 immunizations in various animals have been described for DNA vaccines (reviewed by Liljeqvist and Stahl, 1999). It has been shown that DNA expression plasmids, delivered by intramuscular injection in saline preparation, intravenous administration as liposome-DNA complexes, intranasally using a bacterial vector, oral delivery of microencapsulated DNA or high velocity bombardment of DNA-coated particles into the skin, have proven to be successful in eliciting cytotoxic T lymphocyte responses, T helper responses, and humoral immunity against the encoded antigens.

This technique offers a number of advantages. It is easy and cheap to produce large quantities of pure nucleic acid, and the technique is proven applicable against a wide variety of pathogens. A major advantage, at least where cellular immune responses are desired, is that since the antigen is expressed in the cell, MHC class I presentation of the antigen is easily achieved, which leads to strong cell-mediated immune responses (Svanholm et al., 1999). The flip side, however, is that the antigen may not be as efficiently presented to the immune system for antibody production when it is located inside the cells. Antibody production is better achieved through MHC class II presentation of an extracellular antigen, which results in an enhanced humoral response. Therefore, a nucleic acid vaccine which is more effective in antibody production is desired.

### SUMMARY OF THE INVENTION

In the present invention, we used a DNA vector containing a module to secrete antigens that would normally be expressed inside the cell. The secretion module comprises a signal sequence from a secretory protein, particularly a cytokine, which is linked in frame with the coding sequence of the antigen of interest. The secretion module thus codes for a fusion protein comprising the secretory protein and the antigen, and directs secretion of the fusion protein. The vectors were then used to immunize animals for antibody production. Our results demonstrate that antibody response was significantly enhanced when the secretion module is employed, whereas a DNA vector encoding the same antigen without a secretion module resulted in much less antibody production.

Accordingly, one aspect of the present invention provides a method of generating antibodies to a protein, comprising administering a nucleic acid into an animal wherein the nucleic acid comprises, in a 5′ to 3′ order:

(a) a eukaryotic promoter;

(b) a signal sequence derived from a gene encoding a secretory polypeptide wherein the signal sequence is operatively linked to the promoter; and

(c) a sequence encoding the protein linked in frame with the signal sequence.

In particular, the secretory polypeptide my be a protein which is capable of stimulating an immune response, more particularly a cytokine. The signal sequence is preferably derived from a gene encoding a cytokine selected from the group consisting of granulocyte-macrophage colony stimulating factor (GMCSF), interleukin-2 (IL-2), interleukin-4 (IL-4) and interleukin-12 (IL-12). More preferably, the cytokine is human or murine GMCSF.

In addition to the signal sequence, the nucleic acid may comprise additional sequences from the secretory polypeptide.

This method can be used to generate antibodies against any protein, particularly a protein which is not normally secreted. Preferably, the protein is a microbial protein, such as a bacterial, viral, fungal or parasitic protein.

The nucleic acid may optionally comprises an enhancer to increase expression of the encoded protein.

The nucleic acid may be delivered by any method established in the art, including but not limited to intramuscular, intravenous, intranasal, intradermal, transdermal, subcutaneous, and intraperitoneal administrations.

Another aspect of the present invention provides a method of inducing protective immune response in an animal against a heterologous protein, comprising administering a nucleic acid into the animal wherein the nucleic acid comprises, in a 5′ to 3′ order:

(a) a eukaryotic promoter;

(b) a signal sequence derived from a gene encoding a secretory polypeptide wherein the signal sequence is operatively linked to the promoter; and

(c) a sequence encoding the heterologous protein linked in frame with the signal sequence.

This method can be used as a prophylactic measure or treatment for a disease or medical condition associated with the heterologous protein. Preferably, the heterologous protein is a microbial protein and the method prevents microbial infection.

Another aspect of the present invention provides a nucleic acid comprising, in a 5′ to 3′ order:

(a) a eukaryotic promoter;

(b) a signal sequence derived from a gene encoding a secretory polypeptide wherein the signal sequence is operatively linked to the promoter; and

(c) a sequence encoding the protein linked in frame with the signal sequence.

The nucleic acid is preferably a DNA vector.

Yet another aspect of the present invention provides a pharmaceutical composition useful for eliciting an immune response against a protein, comprising a pharmaceutically acceptable excipient and a nucleic acid comprising, in a 5′ to 3′ order:

(a) a eukaryotic promoter;

(b) a signal sequence derived from a gene encoding a secretory polypeptide wherein the signal sequence is operatively linked to the promoter; and

(c) a sequence encoding the protein linked in frame with the signal sequence.

### BRIEF DESCRIPTION OF THE DRAWINGS

FIG. 1

(A) the schematic of a fusion protein which contains a secretion module using GMCSF sequences, a spacer and an intracellular protein.

(B) the amino acid sequence of the spacer used as an example in the present invention (SEQ ID NO:1). This spacer contains a histidine tag for easy detection, and an enteropeptidase cleavage site which allows separation of the secretion module from the intracellular protein.

(C) the nucleic acid sequence around the spacer (SEQ ID NO:2), showing the 3′ end of GMCSF, the spacer, and the 5′ end of the CAT gene.

FIG. 2

A schematic diagram of 14 constructs harboring various secretion modules and the CAT gene. The “m” preceding GMCSF, IL or EBI means murine. IL-12 p40 and IL-12 p35 are the two subunits of IL-12.

FIG. 3

(A) The plasmids used in each transfection experiment: no plasmid (C, control), CAT only (1), JHE-CAT fusion (2), and human GMCSF-CAT fusion (3).

(B) A Western analysis showing the levels of CAT, GMCSF-CAT and JHE-CAT in the cells or medium from cells transfected with C, 1, 2 or 3.

FIG. 4

FIG. 4 shows the amounts of fusion proteins in the supernatant of cells transfected with the constructs indicated as 1, 2, 3 or 4.

FIG. 5

(A) Western blots probed with antisera produced from mice which were immunized with construct 1, 2, 3 or 4, respectively. “−” indicates that the lane was not loaded with the JHE-411 fusion protein, while “+” indicates that the lane was loaded with the JHE-411 fusion protein.

(B) Schematic of constructs 1, 2, 3 and 4.

FIG. 6

(A) Western blots probed with antisera produced from mice which were immunized with construct 1 or 2, respectively. “−” indicates that the lane was not loaded with the CAT protein, while “+” indicates that the lane was loaded with the CAT protein.

(B) Schematic of constructs 1 and 2.

### DETAILED DESCRIPTION OF THE INVENTION

In order to enhance antibody production, which is more efficient by MHC class II presentation of an extracellular antigen, we used a DNA vector containing a module to secrete antigens that would normally be expressed inside the cell. The secretion module comprises a signal sequence from a secretory protein, particularly a cytokine, which is linked in frame with the coding sequence of the antigen of interest. The secretion module thus codes for a fusion protein of the secretory protein and the antigen, and directs secretion of the fusion protein. Our results demonstrate that antibody response is significantly enhanced when the secretion module is employed, whereas a DNA vector encoding the same intracellular antigen without a secretion module results in much less antibody production.

Prior to describing the invention in further detail, the terms used in this application are defined as follows unless otherwise indicated.

Definitions

The term “protein” refers to a molecule which contains amino acids linked to each other by peptide bonds. As used herein, the term “protein” is not limited by the number of amino acids in the molecule and encompasses peptides, polypeptides and proteins. A protein useful in the present invention contains preferably at least about 5 amino acids, more preferably at least about 10 amino acids, yet more preferably at least about 20 amino acids and most preferably at least about 50 amino acids.

The term “secreting” or “secretion” is the active export of a protein from a cell into the extracellular environment. Generally, secretion occurs through a secretory pathway in the cell, for example, in eukaryotic cells, this involves the endoplasmic reticulum and Golgi apparatus. The term “secretory protein” refers to a protein which is secreted.

The term “signal sequence” refers to a nucleic acid sequence which encodes a signal peptide. A signal peptide, in turn, is a peptide which is recognized by the secretion machinery of the endoplasmic reticulum and Golgi apparatus and is sufficient to result in secretion of the protein which contains the signal peptide. The signal peptide is typically located at the N-terminus of the protein and is cleaved in the process of secretion. Therefore, while a secretory protein is translated with a signal peptide, the final secreted version of the secretory protein usually contains no signal peptide. The signal peptides may differ in amino acid sequences but they all contain 5-10 hydrophobic amino acid residues near the N-terminus. Methods to identify the signal peptide is well established in the art (e.g. see Alberts et al.).

The term “heterologous protein” refers to a protein not naturally encoded by the genome of the host animal.

The term “microbial protein” means a protein found in a microorganism, such as a bacterium, archaebacterium, fungus, virus, protozoan, parasite, alga, slime mold, or prion.

The term “non-secretion competent proteins” means proteins which are not naturally secreted from the cell into the extracellular environment. Examples of non-secretion competent proteins are chloramphenicol acetyl transferase, human immunodeficiency virus (HIV-1) gag, pol, sor, β-galactosidase, c-myc, influenza polymerases PA, PB1, and PB2, Neurospora crassa activator protein, p53 protein, topoisomerases, ecdysone receptor, DNA polymerase subunits, RNA polymerase I, II and III subunits, cytoplasmic and nuclear factors.

The term “promoter” means a DNA sequence which initiates and directs the transcription of a gene into an RNA transcript in cells. A eukaryotic promoter typically contains an AT-rich sequence (the “TATA box”) and is usually found about 30 bases upstream from the transcription initiation site. The promoter useful in the present invention may be any promoter capable of initiating transcription in the host animal. For example, when the host animal is a mammal, mammalian promoters such as the cytomegalovirus (CMV) immediate early promoter, the SV40 large T antigen promoter or the Rous Sarcoma virus (RSV) LTR promoter may be used.

The term “enhancer” means a cis-acting nucleic acid sequence which enhances the transcription of the structural gene and functions in an orientation and position-independent manner. The enhancer can function in any location, either upstream or downstream relative to the promoter. The enhancer may be any DNA sequence which is capable of increasing the level of transcription from the promoter when the enhancer is functionally linked to the promoter, for example the RSV LTR enhancer, baculovirus HR1, HR2 or HR3 enhancers or the CMV immediate early gene product enhancer.

It is also contemplated that the expression of the structural gene may be enhanced by the expression of other factors, for example the IE-1 protein of nuclear polyhedrosis viruses or the herpes simplex virus VP16 transcriptional activator.

The term “functionally linked” or “operatively linked” when describing the relationship between two DNA regions means that they are functionally related to each other and they are located on the same nucleic acid fragment. A promoter is functionally linked to a structural gene if it controls the transcription of the gene and it is located on the same nucleic acid fragment as the gene. An enhancer is functionally linked to a structural gene if it enhances the transcription of that gene and it is functionally located on the same nucleic acid fragment as the gene.

The term “linked in frame” means that one gene is linked at its 3′ end to the 5′ end of a second gene such that after transcription and translation of the genes a single fusion protein comprising the two proteins encoded by the genes is produced. The two genes may also be linked by a spacer nucleic acid sequence.

Methods

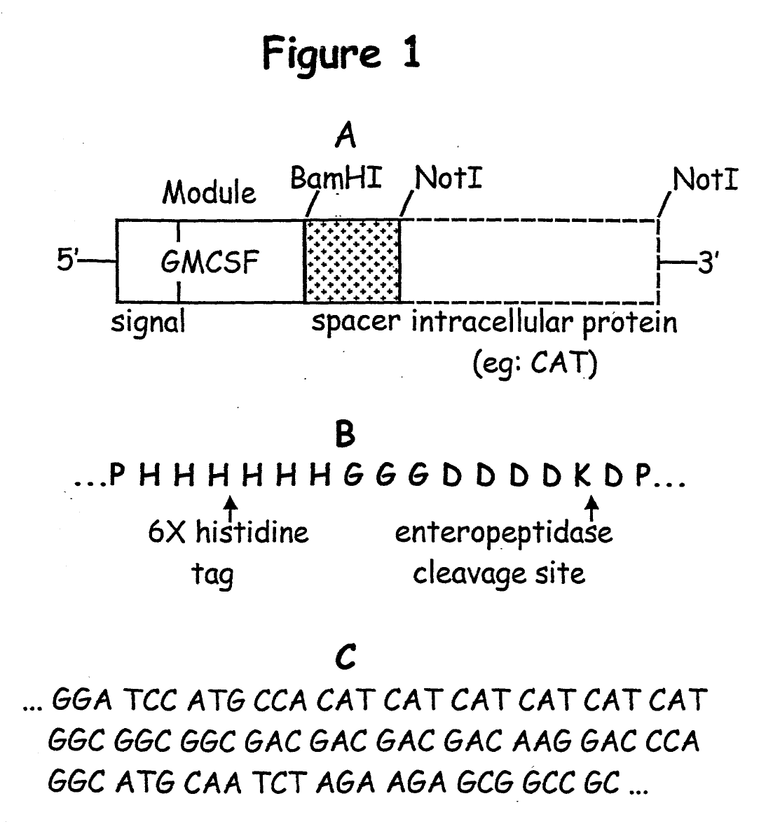

We have previously described modules for the secretion of intracellular proteins from in vitro cultured insect and mammalian cells (U.S. Pat. No. 6,037,150; Farrell et al., 2000). The secretion module encodes a secreted protein lacking a stop codon and a non-secretion competent polypeptides joined in-frame to the 3′ end of the secreted protein (FIG. 1). The secreted protein can be optionally separated from the non-secretion competent polypeptide by a spacer. When placed in an animal expression vector and transfected into appropriate cultured animal cells, a fusion protein is expressed that contains the secreted protein at the N-terminus and the non-secretion competent polypeptide at the C-terminus. The presence of the secreted protein causes the fusion protein to be secreted outside of the cell.

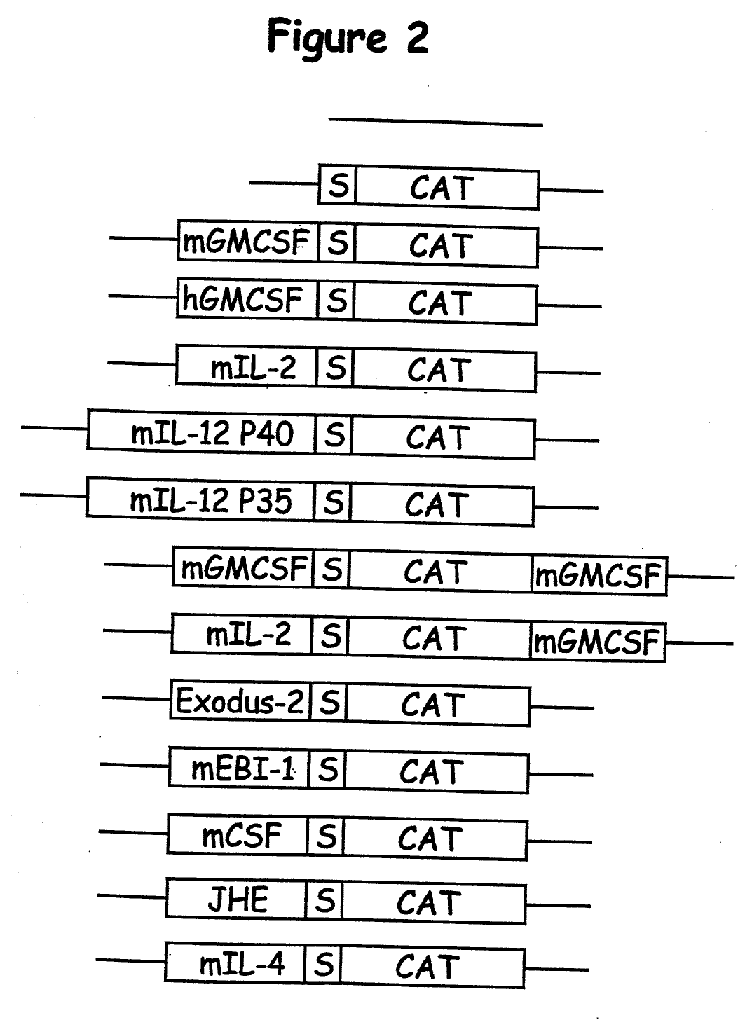

Juvenile hormone esterase (JHE) is employed in U.S. Pat. No. 6,037,150 as the secretory protein to induce secretion of the fusion protein. In addition, a number of other secretory proteins can also be used. FIG. 2 is a schematic of 14 constructs used to demonstrate that a variety of secretion competent polypeptides can be used to secret non-secretion competent polypeptides into an extracellular environment. Thus, the coding sequence of a secretory protein in which the stop codon is deleted was ligated to a spacer and the CAT gene as depicted in FIG. 2. In two of the constructs, the coding sequence for the mature murine GMCSF, without the signal peptide, was also included at the C-terminus of the fusion protein. These constructs were transfected into cultured cells, and supernatant of the cultured cells was collected for CAT assays. All these constructs (except the mCSF-CAT construct which was not tested) gave rise to CAT activities in the supernatant, indicating that the fusion proteins were secreted from the cells, adopted an appropriate conformation and functioned properly.

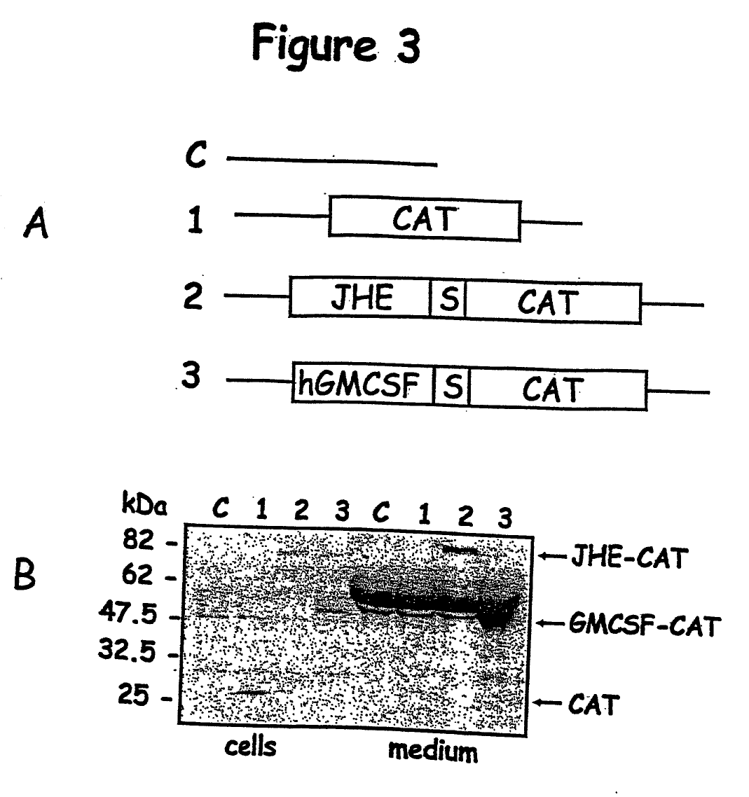

FIG. 3 shows the ability of granulocyte-macrophage colony stimulating factor (GMCSF) to direct secretion of a fusion protein comprising both GMCSF and CAT. Surprisingly, the amount of GMCSF-CAT fusion secreted from the cells was approximately five-fold higher than that of the JHE-CAT fusion, even thought the transfection efficiency for the plasmid encoding GMCSF-CAT was 50% lower than the JHE-CAT expression plasmid.

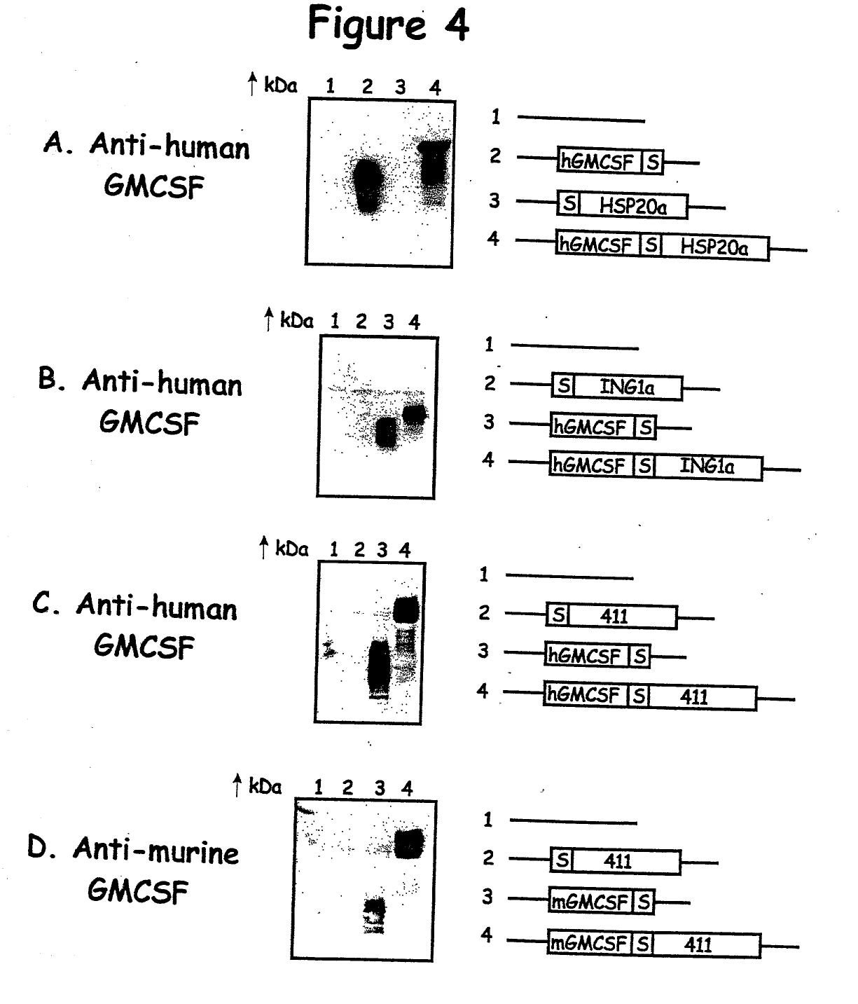

FIG. 4 demonstrates the effect of the secretion modules on various non-secretion competent polypeptides. Thus, coding sequences for heat shock protein 20a, the nuclear protein ING1a or Leishmania chagasi cysteine protease (“411”) were linked in frame at the C-terminus to GMCSF secretion modules and the resulted constructs were introduced into cultured cells. The supernatant was separated in Western blot experiments and probed with anti-GMCSF antibodies. The results show that all the non-secretion competent polypeptides tested were secreted when fused with GMCSF, but not secreted in the absence of the secretion module.

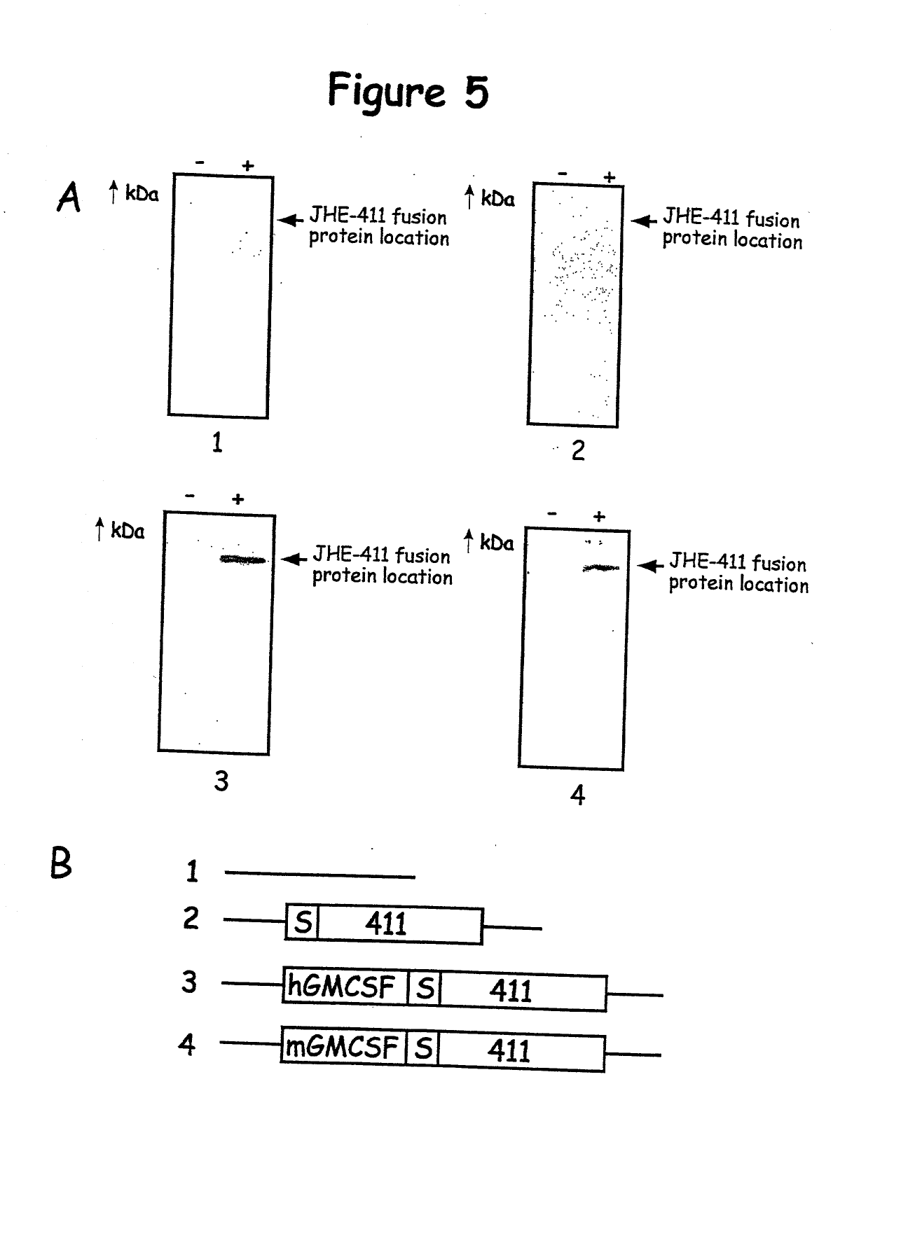

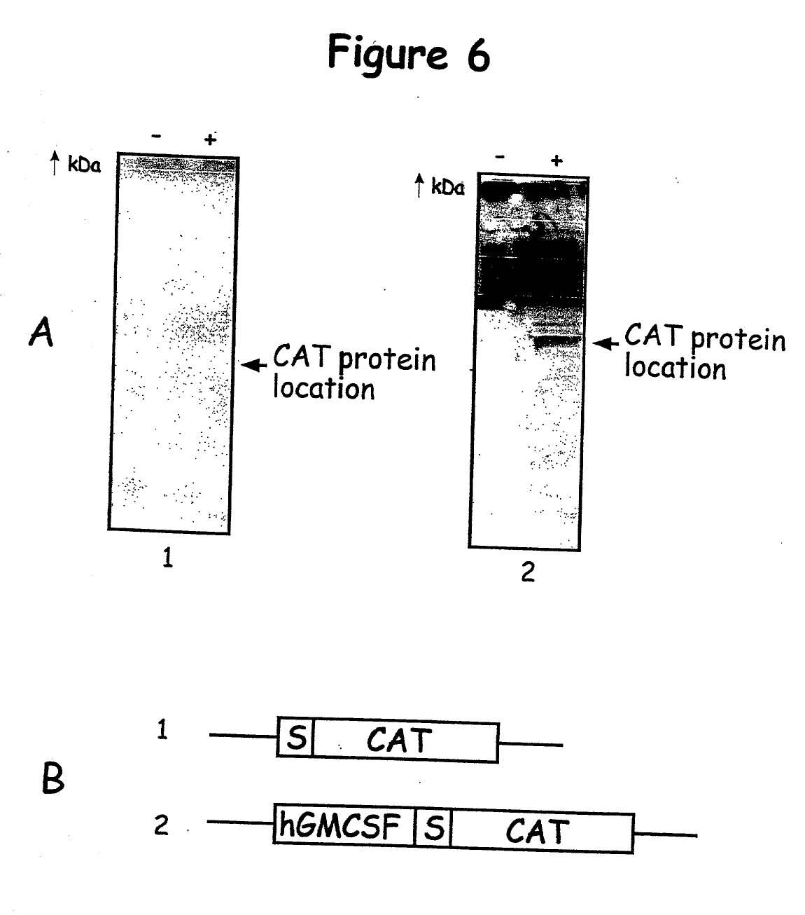

The secretion modules can be used to enhance antibody responses against a protein which is normally located inside the cells. In Example 3, various constructs encoding secretion competent or non-competent proteins (such as 411 or CAT) were injected into mice according to established immunization protocols. Sera were obtained from these mice and used in Western blotting analyses to probe membranes which contain the protein of interest. As shown in FIG. 5, sera obtained from the mice which received plasmids encoding GMCSF-411 fusion proteins were capable of detecting the 411 protein, whereas sera from the mice which received a non-secretion competent 411 construct were entirely negative. Similarly, a GMCSF-CAT construct was effective in inducing CAT-specific antibody production upon being administered as a DNA vaccine, while a CAT construct lacking any secretory protein coding sequence was not (FIG. 6). Therefore, expressing an antigen as a fusion protein with a secretory protein is an effective method for eliciting humoral response. Without being limited to any theory, we believe that secretion of the fusion protein increases the chance of the antigen being encountered by the immune system in blood or lymph. For example, the chance for a secreted antigen to be phagocytosed by a macrophage and presented to leukocytes is much greater than that for an intracellular antigen.

Accordingly, the present invention provides a method of generating antibodies comprising administering a nucleic acid encoding a fusion protein which is rendered secretable by having at least a portion of a secretory protein at its N-terminus. Any secretory protein can be employed in the present invention to direct secretion of the fusion protein. Secretory proteins which themselves stimulate the immune system are preferred.

It is particularly attractive to use a cytokine as the secretory protein, because it is expected that the cytokine will help to stimulate immune responses. Cytokines are well known in the art, including but not limited to IL-1, IL-2, IL-3, IL-4, IL-5, IL-6, IL-7, IL-8, IL-9, IL-10, IL-11, IL-12, IL-13, IL-14, IL-15, IL-16, IL-17, IL-18, IFN-alpha, IFN-beta, IFN-gamma, leukemia-inhibitory factor (LIF), oncostatin M, TGF-beta, TNF-alpha, TNF-beta, macrophage colony stimulating factor (M-CSF), granulocyte colony stimulating factor (G-CSF), and GMCSF. The cytokine is preferably one that is known to enhance antigen presentation, B cell proliferation or T helper cell functions, such as IL-2, IL-4, IL-5, IL-6, IL-10, IL-13, and IFN-gamma. More preferably, the cytokine is GMCSF.

A portion of the secretory protein comprising at least the signal peptide should be included in the fusion protein. When the secretory protein is expected to provide biological activities other than secretion (e.g., a cytokine which may stimulate antibody production), it may be desired to include additional sequences from the secretory protein other than the signal peptide. The amount of additional sequences to be included can be varied depending on the function of the secretory protein. For example, when GMCSF is used as the secretory protein to provide not only the signal peptide but also cytokine function, the portion of GMCSF which is necessary for its cytokine function should be included. In general, however, the fusion protein contains preferably at least about 30%, more preferably at least about 50%, yet more preferably at least about 70%, and most preferably at least about 80% of the amino acid sequence of the secretory protein. It is essential, however, to remove the stop codon from the secretory protein such that the sequence 3′ to the secretory protein can be translated as part of the fusion protein. In a preferred embodiment, the entire secretory protein, less the stop codon, is fused to the non-secretion competent protein of interest with or without a spacer in between.

A promoter is included in the nucleic acid to direct transcription of the coding sequences. Any promoter which is active in the host animal can be used in accordance with the present invention. An enhancer can also be optionally included in the nucleic acid to enhance expression of the fusion protein. For cloning convenience, the nucleic acid may further comprise prokaryotic and/or eukaryotic origin of replication, selectable marker or linker sequences. The nucleic acid may be DNA or RNA, and may be administered as plasmids or fragments.

The present invention further provides a method for eliciting a protective immune response using a nucleic acid encoding the fusion protein as described above as a nucleic acid vaccine. The fusion protein expressed from the nucleic acid would be secreted and exposed to the immune system, thereby inducing antibody production as well as cell-mediated immunity against the antigen, which is part of the fusion protein. As such, the present invention can be used as a prophylactic measure or treatment for a disease or medical condition associated with the particular antigen.

Compositions

The present invention further provides pharmaceutical compositions comprising a nucleic acid encoding the fusion protein as described above and a pharmaceutically acceptable excipient or carrier. Such compositions are useful for immunizing any animal which is capable of initiating an immune response, such as primate, rodent, bovine, ovine, caprine, equine, leporine, porcine, canine and avian species. Both domestic and wild animals may be immunized. The exact formulation of the compositions will depend on the particular fusion protein, the species to be immunized, and the route of administration.

The pharmaceutical composition can be administered intravascularly, transdermally, intraperitoneally, intramuscularly, intradermally, subcutaneously, orally, intranasally (e.g. by aerosol inhalation). Preferably the pharmaceutical composition is administered intramuscularly, intradermally, subcutaneously, or intranasally. Most preferably the pharmaceutical composition is administered intramuscularly. When administered intramuscularly, the nucleic acid is preferably injected in a saline preparation. The nucleic acid may be administered intravenouly as micelles or liposome-DNA complexes, intranasally as an aerosol, orally as microencapsulated DNA, or intradermally in DNA-coated particles by high velocity bombardment. Suitable formulations for use in the present invention can be found in Remington's Pharmaceutical Sciences.

The pharmaceutical composition may optionally comprise an adjuvant or a combination of adjuvants. Such adjuvants may include, but are not limited to, Freunds complete adjuvant, Freunds incomplete adjuvant, aluminum hydroxide, dimethyldioctadecyl-ammonium bromide, Adjuvax (Alpha-Beta Technology), Inject Alum (Pierce), Monophosphoryl Lipid A (Ribi Immunochem Research), MPL+TDM (Ribi Immunochem Research), Titermax (CytRx), QS21, the CpG sequences (U.S. Pat. No. 6,194,388), toxins, toxoids, glycoproteins, lipids, glycolipids, bacterial cell walls, subunits (bacterial or viral), carbohydrate moieties (mono-, di-, tri-, tetra-, oligo- and polysaccharide), various liposome formulations or saponins. The adjuvant is preferably the CpG sequences which may be conveniently included in the nucleic acid of the present invention.

The following examples are offered to illustrate this invention and are not to be construed in any way as limiting the scope of the present invention.

### EXAMPLES

In the examples below, the following abbreviations have the following meanings. Abbreviations not defined have their generally accepted meanings.

| | |
|---|---|
| ° C. | degree Celsius |
| hr | hour |
| min | minute |
| μM | micromolar |
| mM | millimolar |
| M | molar |
| ml | milliliter |
| μl | microliter |
| mg | milligram |
| μg | microgram |
| PAGE | polyacrylamide gel electrophoresis |
| rpm | revolutions per minute |
| FBS | fetal bovine serum |
| DTT | dithiothrietol |
| SDS | sodium dodecyl sulfate |
| PBS | phosphate buffered saline |
| DMEM | Dulbecco's modified Eagle's medium |
| α-MEM | α-modified Eagle's medium |
| β-ME | β-mercaptoethanol |
| CMV | cytomegalovirus |
| LTR | long terminal repeat |
| DMSO | dimethylsulfoxide |
| IL | interleukin |
| CSF | colony stimulating factor |
| GMCSF | granulocyte-macrophage colony stimulating factor |
| TNF | tumor necrosis factor |
| TGF | transforming growth factor |
| CAT | chloramphenical acetyl transferase |
| JHE | juvenile hormone esterase |
| ORF | open reading frame |

### Example 1

FIG. 2 is a schematic of 14 constructs used to demonstrate that a variety of secretion competent polypeptides can be used to secrete non-secretion competent polypeptides such as chloramphenicol acetyl transferase (CAT) into an extracellular environment. These chimeric genes were inserted into the multiple cloning site of the mammalian expression vector pCDNA3.1+ (Invitrogen) that employs the Cytomegalovirus promoter-enhancer to drive foreign gene expression. This same vector is used in DNA vaccinations of mammals.

The vector pCDNA3.1.CAT was constructed by digesting the plasmid pBmA.CAT (Johnson et al., 1992) with BamHI and ligating the 0.78 kbp fragment containing the CAT open reading frame (ORF) into the unique BamHI site of pCDNA3.1.

The vector pCDNA3.1.JHE.His.CAT was constructed as follows. Digesting pJHE(6HEP).cat (Farrell et al., 2000) partially with BamHI and completely with NotI released a 2.46 kb fragment containing the JHE-CAT fusion protein ORF. This was ligated into the unique BamHI/NotI site of pcDNA3.1 to yield pCDNA3.1.JHE.His.CAT

The vector pcDNA3.1.His.CAT containing the utility spacer region linked in frame to the CAT gene was constructed by digesting pCDNA3.1.JHE.His.CAT with BamHI to remove the JHE ORF and self-ligating the remaining vector to yield pcDNA3.1.His.CAT. A synthetic start codon present an the 5′ end of the utility spacer region ensures that the ORF is translated correctly.

The vector pcDNA3.1.hGMCSF.His.CAT was generated by ligating the 0.43 kb BamHI fragment from pIE1/153A.gmcsf(6hep)cat (Farrell et al., 2000) into the unique BamHI site of pcDNA3.1.His.CAT to yield pcDNA3.1.hGMCSF.His.CAT.

The vector pcDNA3.1.mGMCSF.His.CAT was generated in several steps. First, appropriate mutagenic PCR primers (containing Bam HI sites) were synthesized and used to amplify a 0.44 kbp product containing murine granulocyte-macrophage colony stimulating factor (GM-CSF; Gough et al., 1985) with no stop codon using a cDNA template from reverse transcribed mouse liver mRNA. This PCR product was digested with BamHI and ligated into to unique BamHI site of the vector pcDNA3.1.His.CAT to yield pcDNA3.1.mGMCSF.His.CAT.

The vector pcDNA3.1.His was constructed in several steps. First, appropriate mutagenic PCR primers were synthesized and used to amplify a 1.72 kbp product containing Heliothis viriscens juvenile hormone esterase (JHE; Hammock et al., 1990) with no stop codon using a pIE1/153A.jhe (Farrell et al., 1998) as a template. This PCR product was digested with BamHI and ligated into the unique BamHI site of the vector p6HEP(SphI) (Farrell et al., 2000) to yield pjhe.6HEP(SphI).

A potential stop codon in pjhe.6HEP(SphI) was corrected as follows. Appropriate PCR primers were synthesized and used to amplify a 1.58 kbp product using a pjhe.6HEP(SphI) as a template that was digested with XbaI. Then the vector pjhe.6HEP(SphI) was digested with XbaI to release a 1.58 kbp fragment and ligated with the XbaI digested PCR product to yield pjhe.6HEP(SphI, corrected). The vector pjhe.6HEP(SphI, corrected) was digested partially with BamHI and completely with NotI to release a 1.68 kbp fragment containing the JHE gene linked in-frame to a histidine tag and enteropeptidase cleavage site that was ligated into the unique BamHI/NotI site of pcDNA3.1+ (Invitrogen) to yield pcDNA3.1.JHE.His. The vector pcDNA3.1.JHE.His was then digested with BamHI to release the JHE gene and self ligated to yield pCDNA3.1.His.

The vector pCDNA3.1.His.CAT(no stop) was generated in several steps. PCR amplification using appropriate primers and platinum Taq polymerase (Life Technologies) from pBmA.cat (Johnson et al., 1992) yielded a 0.67 kb product containing the CAT ORF without a stop codon. This PCR product was digested with NotI and ligated into the unique NotI site of pCDNA3.1.His to yield pCDNA3.1.His.CAT(no stop).

The vector pCDNA3.1.IL2.His.CAT (stop) was generated in several steps. PCR amplification using appropriate primers and platinum Taq polymerase (Life Technologies) from pBmA.cat yielded a 0.67 kb product containing the CAT ORF with a stop codon. This PCR product was digested with NotI and ligated into the unique NotI site of pCDNA3.1His to yield pCDNA3.1.His.CAT(stop). Next, appropriate mutagenic PCR primers (containing Bam HI sites) were synthesized and used to amplify a 0.52 kbp product containing murine interleukin 2 (IL-2; Kashima et al., 1985) with no stop codon using a cDNA template from reverse transcribed mouse liver mRNA. This PCR product was digested with BamHI and ligated into to unique BamHI site of the vector pcDNA3.1.His.CAT(stop) to yield pcDNA3.1.mIL2.His.CAT.

The vector pCDNA3.1.mIL12p35.His.CAT (stop) was generated in several steps. First, appropriate mutagenic PCR primers (containing Bam HI sites) were synthesized and used to amplify a 0.67 kbp product containing the p35 subunit of murine interleukin 12 (IL-12; Schoenhaut et al., 1992) with no stop codon using a cDNA template from reverse transcribed mouse liver mRNA. This PCR product was digested with BamHI and ligated into to unique BamHI site of the vector pcDNA3.1.His.CAT(stop) to yield pcDNA3.1.mIL12p35.His.CAT.

The vector pCDNA3.1.mIL12p40.His.CAT (stop) was generated in several steps. First, appropriate mutagenic PCR primers (containing Bam HI sites) were synthesized and used to amplify a 1.02 kbp product containing the p40 subunit of murine interleukin 12 (IL-12; Schoenhaut et al., 1992) with no stop codon using a cDNA template from reverse transcribed mouse kidney mRNA. This PCR product was digested with BamHI and ligated into to unique BamHI site of the vector pcDNA3.1.His.CAT(stop) to yield pcDNA3.1.mIL12p35.His.CAT.

The vector pCDNA3.1.mGMCSF.His.CAT.mGMCSF (stop) was generated in several steps. First, appropriate mutagenic PCR primers (containing XhoI sites) were synthesized and used to amplify a 0.39 kbp product containing murine GM-CSF without signal peptide coding using the plasmid pCDNA3.1.mGMCSF.His as a template. This PCR product was digested with ApaI and ligated into to unique XhoI site of the vector pcDNA3.1.mGMCSF.His.CAT(no stop) to yield pcDNA3.1.mGMCSF.His.CAT.mGMCSF.

The vector pCDNA3.1.IL2.His.CAT.mGMCSF (stop) was generated as follows. The vector pcDNA3.1.mGMCSF.His.CAT.mGMCSF was digested with BamHI to remove the N-terminal murine GM-CSF and the linearised vector, pcDNA3.1.His.CAT.mGMCSF, was ligated with the 0.50 kbp BamHI fragment from pCDNA 3.1.IL2.His.CAT (stop) containing the murine IL-2 gene and lacking a stop codon to yield pCDNA3.1.IL2.His.CAT.mGMCSF (stop).

The vector pCDNA3.1.Exodus-2.His.CAT (stop) was generated in several steps. First, appropriate mutagenic PCR primers were synthesized and used to amplify a 0.42 kbp product containing the Exodus-2 open reading frame (Hromas et al., 1997) with no stop codon using a cDNA template from reverse transcribed mouse spleen mRNA. This PCR product was digested with both BamHI and HindIII and ligated into to unique BamHI/Hind III site of the vector pcDNA3.1.His.CAT(stop) to yield pcDNA3.1.Exodus-2.His.CAT.

The vector pCDNA3.1.mEBI-1.His.CAT (stop) was generated in several steps. First, appropriate mutagenic PCR primers were synthesized and used to amplify a 0.35 kbp product containing the mEBI-1 (Ngo et al., 1998) with no stop codon using a cDNA template from reverse transcribed mouse spleen mRNA. This PCR product was digested with both BamHI and HindIII and ligated into to unique BamHI/Hind III site of the vector pcDNA3.1.His.CAT(stop) to yield pcDNA3.1.mEBI-1.His.CAT.

The vector pCDNA 3.1.mIL4.His.CAT (stop) was generated in several steps. First, appropriate mutagenic PCR primers (containing Bam HI sites) were synthesized and used to amplify a 0.43 kbp product containing murine interleukin 4 (IL-4; Sideras et al., 1987) with no stop codon using a cDNA template from reverse transcribed mouse spleen mRNA. This PCR product was digested with BamHI and ligated into to unique BamHI site of the vector pcDNA3.1.His.CAT(stop) to yield pcDNA3.1.mIL4.His.CAT.

Liposome mediated transfection of Chinese Hamster Ovary (CHO) cells was used to demonstrate that a variety of secretion competent polypeptides can be used secrete an intracellular protein such as chloramphenicol acetyl transferase (CAT) into an extracellular environment. CHO cells were transfected with Lipofectin (Life Technologies) according to the manufacturers instructions and the supernatant was harvested and analyzed 3 days post-transfection and analyzed by Western blotting using a polyclonal antibody recognizing the CAT protein (5 Prime-3 Prime). The results of the Western Blot are shown in FIG. 3. The results demonstrate that CHO cells transfected with expression constructs containing a secretion-competent polypeptide (eg: JHE or human GM-CSF) linked to the CAT gene were able to secrete CAT into the supernatant, as shown by a strong immunoreactive band indicating the presence of the CAT protein. CHO cells transfected with an expression construct containing only the CAT gene were unable to efficiently direct CAT into the supernatant due to the absence of any immunoreactive band for the CAT protein. In the latter case, some CAT may occasionally be detected in the supernatant due to passive leakage of CAT from animal cells.

### Example 2

FIG. 4 contains a schematic of several constructs used to demonstrate that secretion competent polypeptides can be used to secrete intracellular proteins other than CAT from animal cells into an extracellular environment such as heat shock protein p25 (Bany and Schultz, 2001), the A exon of human ING1 protein (Ma et al., 1999) and the Leshmania cysteine protease (411, Opyene and Gedamu, 1997). These chimeric genes were inserted into the multiple cloning site of the mammalian expression vector pCDNA3.1+ (Invitrogen) that employs the Cytomegalovirus promoter-enhancer to drive foreign gene expression. This same vector is used in DNA vaccinations of mammals.

The vector pCDNA3.1.mIL2.His.ING1a was generated in several steps. First, appropriate mutagenic PCR primers (containing Xba I sites) were synthesized and used to amplify a 0.57 kbp product containing the coding sequence for the A exon from the human ING protein (Ma et al., 1999). The vector pcDNA3.1.mIL2.His.CAT was digested with XbaI to remove the C-terminal CAT gene and the linearized vector pcDNA3.1.mIL2.His was ligated with the ING1a PCR product that was digested with XbaI to yield pCDNA3.1.mIL2.His.ING1a.

The vector pCDNA3.1.His.ING1a was generated by digesting pCDNA3.1.mIL2.His.ING1a with BamHI to remove the murine IL-2 gene and self-ligating the open vector to yield pCDNA3.1.His.ING1a.

The vector pCDNA3.1.hGMCSF.His.ING1a was generated by ligating the 0.43 kbp BamHI fragment from pIE1/153A.gmcsf(6hep)cat (Farrell et al., 2000) containing the human GM-CSF gene lacking a stop codon into the unique BamHI site of pCDNA3.1.His.ING1a to yield pcDNA3.1.hGMCSF.His.ING1a.

The vector pCDNA3.1.hGMCSF.His.HSP20a was generated in several steps. First, appropriate mutagenic PCR primers (containing Xba I sites) were synthesized and used to amplify a 0.65 kbp fragment containing the murine HSP20-like protein gene (Bany and Schultz, 2001), encoding a putative nuclear factor. This PCR product was digested with XbaI and ligated into to unique XbaI site of pcDNA3.1.hGMCSF.His to yield pcDNA3.1.hGMCSF.His.HSP20a.

The vector pCDNA3.1.His.HSP20a was generated by digesting pcDNA3.1.hGMCSF.His.HSP20a with BamHI to remove the human GM-CSF gene and self-ligating the open vector to yield pCDNA3.1.His.HSP20a.

The vector pcDNA3.1.hGMCSF.His.411 was generated in several steps. First, appropriate mutagenic PCR primers (containing Not I sites) were synthesized and used to amplify a product containing coding for a Leishmania chagasi cysteine protease using pLdccys1 cDNA clone (Opyene and Gedamu, 1997) as a template. This PCR product was digested with NotI and cloned into the unique NotI site of pcDNA3.1.hGMCSF.His to yield pcDNA3.1.hGMCSF.His.411.

The vector pcDNA3.His.411 was generated by digesting pcDNA3.1.hGMCSF.His.411 with BamHI to remove the human GM-CSF gene and self-ligating the open vector to yield pCDNA3.1.His.411.

The vector pcDNA3.1.mGMCSF.His.411 was generated by digesting the vector pcDNA3.1.hGMCSF.His.411 with BamHI to remove the human GM-CSF gene and ligating the linearized vector with the 0.44 kbp BamHI fragment from pcDNA3.1,mGMCSF.His containing the murine GM-CSF gene lacking a stop codon to yield pcDNA3.1.mGMCSF.His.411.

These plasmids were used to express the encoded proteins in cultured cells, and the supernatant was collected to determine if the various non-secretion competent proteins were secreted. The supernatant was resolved by electrophoresis, transferred to membranes, and probed with anti-GMCSF antibodies which were capable of detecting the fusion proteins. In the supernatant of every cell which was transfected with a secretion module-containing plasmid, a positive signal appeared (FIG. 4), indicating that the secretion module was effective in causing various non-secretion competent proteins to be secreted.

### Example 3

In the previous two examples the secretion of intracellular proteins or protein subunits using secretion competent polypeptides fused to them has been demonstrated in in vitro cultures of animal cells. This technique can also be used in vivo in DNA vaccines to enhance the immune response against a non-secretion competent polypeptide antigen. Mammalian expression plasmids can be injected into the muscles of mammals such as mice or humans where they are taken up into the muscle cells. Once inside the cells, the encoded protein contained in the expression plasmids is expressed under control of a mammalian promoter. If the encoded protein is a fusion protein containing a secretion competent polypeptide functionally linked to non-secretion competent polypeptide, the chimeric protein will be secreted into the blood where it will be recognized as foreign by the animal's immune system and an immune response against the non-secretion competent polypeptide will result. It is expected that the immune response against the non-secretion competent polypeptide will be enhanced because it is secreted from the muscle cells. To demonstrate this, mice were injected with mammalian expression plasmids containing the genes encoding the intracellular proteins bacterial CAT or the Leishmania cysteine protease linked to secretion competent polypeptides.

Six to eight week old female BALB/c mice were used for DNA immunizations. Eight groups of four mice were injected with different plasmid constructs. Fifty micrograms of endotoxin-free plasmid DNA was diluted in PBS in a total volume of 50 microliters. For immunization, mice were injected into the thigh muscle on days 0, 14, 21 and 27. Sera samples were collected from the ocular vein prior to every injection, clotted overnight at 4° C. to remove haemocytes and stored at −20° C. for later analysis.

Analysis of sera samples was performed by Western blotting. Various positive and negative control protein samples and molecular mass markers were resolved using SDS-polyacrylamide gel electrophoresis and transferred by electroblotting to either nitrocellulose or nylon membranes. After transfer, membranes were cut into strips which were blocked for 1 h at room temperature in PBS-0.1% Tween-20 (PBST) containing 10% (w/v) skim milk powder (PBSTM). Then each strip was incubated overnight at room temperature with a small volume of PBST containing the various samples of mouse sera at a dilution of 100:1. The strips were washed twice for 15 min with PBST, and incubated for 2 h with PBSTM containing horseradish peroxidase-conjugated goat anti-mouse IgG. After washing twice with PBST, the strips were incubated with ECL chemiluminescent substrate (Amersham) according to the supplier's instructions and exposed to X-ray film.

FIG. 5 shows Western blots of membrane strips probed with various sera samples from DNA immunized mice. Each strip contains two lanes of positive and negative control protein samples. The positive control lane containing the protein Leishmania chagasi cysteine protease is fifty microliters of cell culture supernatant from insect cells transfected with plasmid pIE1/153A.JHE.411 and incubated for 3 days following transfection. The protein resulting from this transfection is a fusion protein containing juvenile hormone esterase (Hammock et al., 1990) on the N-terminus and Leishmania chagasi cysteine protease on the C-terminus. The negative control is fifty microliters of cell culture supernatant from insect cells transfected with plasmid pIE1/153A and incubated for 3 days following transfection. Representative sera samples from each group of immunized mice used in the Western blotting show that the Leishmania chagasi cysteine protease could be detected by antibodies present in the sera of mice immunized with pcDNA3.1.hGMCSF.His.411 and pcDNA3.1.mGMCSF.His.411 but not pcDNA3.1.His.411 or control samples. This indicates that the humoral immune response against the Leishmania chagasi cysteine protease, an intracellular protein, was enhanced because this protein was secreted from the muscle cells of mice as a fusion protein with either human or murine GM-CSF as a secretion competent polypeptide.

FIG. 6 shows Western blots of membrane strips probed with various sera samples from DNA immunized mice. Each strip contains two lanes of positive and negative control protein samples. The positive control lane containing the bacterial enzyme chlorampheniocol acetyl transferase (CAT) is fifty microliters of cell culture supernatant from Bombyx mori (Bm5) insect cells infected with the recombinant baculovirus BmNPV.p25.CAT and incubated for 5 days following infection. The negative control is fifty microliters of cell culture supernatant from Bm5 cells infected with the wild type baculovirus (BmNPV). Representative sera samples from each group of immunized mice used in the Western blotting show that the CAT protein could be detected by antibodies present in the sera of mice immunized with pcDNA3.1.hGMCSF.His.CAT but not pcDNA3.1.His.CAT. This indicates that the humoral immune response against CAT, an intracellular protein, was enhanced because this protein was secreted from the muscle cells of mice as a fusion protein with human GM-CSF as a secretion competent polypeptide.

We claim:

1. A method of generating antibodies to a protein, comprising administering a nucleic acid into an animal wherein the nucleic acid comprises, in a 5′ to 3′ order:
(a) a eukaryotic promoter;
(b) a signal sequence derived from a gene encoding a cytokine wherein the signal sequence is operatively linked to the promoter; and
(c) a sequence encoding the protein linked in frame with the signal sequence.

2. The method of claim 1 wherein the nucleic acid further comprises additional sequences from the gene encoding the cytokine.

3. The method of claim 1 wherein the cytokine is selected from the group consisting of GMCSF, IL-2, IL-4 and IL-12.

4. The method of claim 1 wherein the cytokine is human or murine GMCSF.

5. The method of claim 1 wherein the protein is a microbial protein.

6. The method of claim 1 wherein the nucleic acid further comprises an enhancer.

7. The method of claim 1 wherein the nucleic acid is administered intradermally, intramuscularly, intranasally or orally.

8. The method of claim 1 further comprising administration of an adjuvant.

9. The method of claim 1 wherein the sequence encoding the protein encodes at least about 5 amino acids.

10. A method of inducing a protective immune response in an animal against a heterologous protein, comprising administering a nucleic acid into the animal wherein the nucleic acid comprises, in a 5′ to 3′ order:
(a) a eukaryotic promoter;
(b) a signal sequence derived from a gene encoding a cytokine wherein the signal sequence is operatively linked to the promoter; and
(c) a sequence encoding the heterologous protein linked in frame with the signal sequence.

11. The method of claim 10 wherein the nucleic acid further comprises additional sequences from the gene encoding the cytokine.

12. The method of claim 10 wherein the cytokine is selected from the group consisting of GMCSF, IL-2, IL-4 and IL-12.

13. The method of claim 10 wherein the cytokine is human or murine GMCSF.

14. The method of claim 10 wherein the heterologous protein is a microbial protein.

15. The method of claim 10 wherein the nucleic acid further comprises an enhancer.

16. The method of claim 10 wherein the nucleic acid is administered intradermally, intramuscularly, intranasally or orally.

17. The method of claim 10 which results in a prophylactic vaccination, amelioration, or treatment of a disease or medical condition associated with the heterologous protein.

18. The method of claim 10 further comprising administration of an adjuvant.

19. The method of claim 10 wherein the sequence encoding the heterologous protein encodes at least about 5 amino acids.

20. A nucleic acid encoding a fusion protein, comprising, in a 5′ to 3′ order:
(a) a eukaryotic promoter;
(b) a signal sequence derived from a gene encoding a cytokine wherein the signal sequence is operatively linked to the promoter; and
(c) a sequence encoding a non-secretion competent protein linked in frame with the signal sequence.

21. The method of claim 20 wherein the nucleic acid further comprises additional sequences from the gene encoding the cytokine.

22. The nucleic acid of claim 20 wherein the nucleic acid is a DNA vector.

23. A pharmaceutical composition useful for eliciting an immune response against a protein, comprising a pharmaceutically acceptable excipient and the nucleic acid of claim 20.

24. The pharmaceutical composition of claim 23 wherein the cytokine is selected from the group consisting of GMCSF, IL-2, IL-4 and IL-12.

25. The pharmaceutical composition of claim 23 wherein the cytokine is human or murine GMCSF.

26. The pharmaceutical composition of claim 23 wherein the non-secretion competent protein is a microbial protein.

27. The pharmaceutical composition of claim 23 further comprising an adjuvant.

28. The pharmaceutical composition of claim 23 wherein the sequence encoding the non-secretion competent protein encodes at least about 5 amino acids.
